(19) United States
(12) Patent Application Publication
(10) Pub. No.: US 2008/0275531 A1
(43) Pub. Date: Nov. 6, 2008

(54) IMPLANTABLE HIGH EFFICIENCY DIGITAL STIMULATION DEVICE

(76) Inventors: Cherik Bulkes, Sussex, WI (US); Stephen Denker, Mequon, WI (US)

Correspondence Address: QUARLES & BRADY LLP, 411 E. WISCONSIN AVENUE, SUITE 2040, MILWAUKEE, WI, 53202-4497, US

(21) Appl. No.: 12/114,564

(22) Filed: May 2, 2008

Related U.S. Application Data
(60) Provisional application No. 60/915,981, filed on May 4, 2007.

(51) Int. Cl. A61N 1/05

(52) U.S. Cl. 607/62

(57) ABSTRACT

An implantable device provides artificial electrical stimulation of animal tissue using a plurality of electrodes. A sensing unit detects a physiological parameter at a stimulation site. A control unit governs the stimulation, in response to the detected physiological parameter, by selecting certain pairs of the electrodes and by defining the shape, duration, and duty cycle of a segmented stimulation waveform. A stimulation signal generator produces the segmented stimulation waveform that has a first segment and a second segment that with respect to the first segment is longer in duration lesser in magnitude and opposite in polarity.

### CROSS-REFERENCE TO RELATED APPLICATIONS

This application claims benefit of U.S. Provisional Patent Application No. 60/915,981 filed May 4, 2007.

### STATEMENT REGARDING FEDERALLY SPONSORED RESEARCH OR DEVELOPMENT

Not Applicable

### BACKGROUND OF THE INVENTION

1. Field of Invention

The present invention relates to implantable medical devices which deliver energy to stimulate tissue in an patient, and more particularly to highly efficient stimulation devices that use digital stimulation output for use in a medical device that is implanted adjacent to tissue or organ.

2. Description of the Related Art

A remedy for people with slowed or disrupted natural heart activity is to implant a cardiac pacing device which is a small electronic apparatus that stimulates the heart to beat at regular rates. The size of the implant is very much a function of the required energy to be stored. However, a traditional method of minimizing current drain from the battery is the use of high impedance leads, which have inherent I²*R losses, for which ironically no benefit is received, other than reduction of maximum current. What should be sought is a reduction of energy consumption, not absolute current. The consumed energy is proportional to the current, multiplied by the applied voltage, multiplied by the duration. In our embodiment we will show that by minimizing I²*R losses and by minimizing overall duration and a novel stimulation waveform, the overall energy consumption is reduced and by doing so enabling methods of stimulation, previously impractical due high power consumption. The overall effect is expected to be at least a factor of two for conventional pacing (2 . . . 5Volt) and up to an order of magnitude or more for higher voltage applications (10 . . . 50Volt).

Typically the stimulation device is implanted in an patient's chest and has sensor electrodes that detect electrical impulses associated with in the heart contractions. These sensed impulses are analyzed to determine when abnormal cardiac activity occurs, in which event a pulse generator is triggered to produce electrical pulses. Wires carry these pulses to electrodes placed adjacent specific cardiac muscles, which when electrically stimulated contract the heart chambers. It is important that the stimulation electrodes be properly located to produce contraction of the heart chambers.

Modern cardiac pacing devices vary the stimulation to adapt the heart rate to the patient's level of activity, thereby mimicking the heart's natural activity. The pulse generator modifies that rate by tracking the activity of the heart or by responding to other sensor signals that indicate body motion or respiration rate.

U.S. Published Patent Application No. 2008/0077184 describes an apparatus provided for artificially stimulating internal tissue of an patient by means of an implanted medical device adapted for implantation into the patient's blood vasculature. The implanted medical device comprises a power supply and first and second stimulation electrodes for contacting the tissue. A control unit governs operation of a stimulation signal generator connected to the first and second stimulation electrodes. The stimulation signal generator produces a series of electrical stimulation pulses and a selectable voltage intensifier increases the voltage, for applications requiring higher voltages such as transvascular neural stimulation, of each electrical stimulation pulse to produce an output pulse that is applied to the first and second stimulation electrodes. One version of the medical device includes a mechanism that is connected to the first and second stimulation electrodes for sensing physiological effects from the electrical stimulation pulse and producing a feedback signal indicating such effects. Although this stimulation apparatus offered several advantages over other types of stimulators, it required energy efficient systems and highly robust signal sensing to be developed. Such energy efficient stimulation systems meet clinical needs of implanted stimulation devices with internal or external energy sources. An energy efficient stimulation system minimizes requirements for battery recharging or replacement for external powered systems and it improves the battery lifetime of internally powered systems. It's efficiency may also enable therapies that are not practical with current systems. Moreover, robust signal sensing will improve the signal detection and signal analysis tasks due to very low artifact content in the sensed signal.

### SUMMARY OF THE INVENTION

An implantable apparatus is provided for artificial electrical stimulation of tissue in a patient. That apparatus comprises a plurality of electrodes in contact with the tissue at one or more locations inside the patient and a power supply that furnishes energy for the artificial electrical stimulation. A control unit governs the artificial electrical stimulation by programmable selection of at least some of the plurality of electrodes. A stimulation signal generator, connected to the control unit, produces a segmented stimulation waveform that has positive segments, pause segments and negative segments, wherein the shape, duration and duty cycle of the segmented stimulation waveform is defined by the control unit.

In a preferred embodiment, the apparatus also comprises a voltage intensifier connected to stimulation signal generator for increasing the voltage of the segmented stimulation waveform and produce an output waveform that is applied to selected electrodes. The voltage intensifier may be a flying capacitor-type voltage doubler, bipolar mode voltage doubler, or a combination of a flying capacitor type and a bipolar mode voltage doubler.

A preferred segmented stimulation waveform has a first segment with a first amplitude, and a second segment that is longer in duration than the first segment and that has a second amplitude which is opposite in polarity and lesser in absolute magnitude to the first amplitude.

An embodiment of the novel apparatus includes sensing unit for detecting at least one physiological parameter of the patient. That sensing unit comprises an electrode pair for implantation inside the patient, an instrumentation amplifier having an internal voltage reference, a passive filter coupling the electrode pair to the instrumentation amplifier.

### BRIEF DESCRIPTION OF DRAWINGS

FIG. 1 shows the anatomical references of the possible stimulation sites of the vagal nervous system in the fat pads of the epicardium;

FIG. 2 is a block schematic diagram of the electrical circuitry for a stimulation module according to the present invention;

FIG. 3 is a schematic diagram of a voltage intensifier in the intravascular medical device; and

FIG. 4 is a schematic diagram of a voltage inverter;

FIG. 5 illustrates a control unit output signal applied to a voltage doubler to increase the amplitude of the output signal;

FIG. 6 depicts waveform diagrams related to bipolar stimulation signal generation;

FIG. 7 is a schematic diagram of high level modules in one embodiment of the stimulation system;

FIG. 8 is a schematic diagram of high level modules in another embodiment of the stimulation system;

FIG. 9 is an equivalent circuit diagram of the stimulation leads and tissue;

FIGS. 10A and 10B show conventional unipolar and bipolar waveforms in use by prior stimulation devices;

FIG. 11 depicts one period of a composite stimulation pulse produced by the present stimulation system;

FIGS. 12A and B depict one period of an alternative composite stimulation pulse and a multi-lobe composite pulse;

FIG. 13 is a schematic diagram of a sensing amplifier with an internal reference and a high pass filter to reject DC and low frequency signals;

FIG. 14 shows the frequency response of the band pass filtering used in the stimulation system;

FIG. 15 is a schematic diagram of a sensing amplifier that has an internal reference and signal pre-filters;

FIG. 16 shows the details of the internal reference;

FIG. 17A shows exemplary waveforms at various nodes in a derivative zero transition detector schematically shown in FIG. 17B; and

FIG. 18A shows a hysteresis waveform in another derivative zero transition detector schematically shown in FIG. 18B.

### DETAILED DESCRIPTION OF THE INVENTION

An apparatus, comprising a highly efficient stimulator with digital output, can be employed to stimulate simultaneously one or more other areas of human physiology as shown in subsequent descriptions and examples. The stimulator may be implanted subcutaneously or intravascularily in the body to stimulate heart muscle, organ nerves, such as the cardiac vagal nerve which can be stimulated transvenously e.g. from the inferior vena cava or coronary sinus. In addition to cardiac applications, the stimulation apparatus can provide neural stimulation, for example treatment of Parkinson's disease or obsessive/compulsive disorder. The electrical stimulation also may be applied to muscles, the spine, the gastro/intestinal tract, the pancreas, and the sacral nerve. The apparatus may also be used for GERD treatment, endotracheal stimulation, pelvic floor stimulation, treatment of obstructive airway disorder and apnea, molecular therapy delivery stimulation, chronic constipation treatment, and electrical stimulation for bone healing. The current invention can provide stimulation for two or more clinical purposes simultaneously as will be described later.

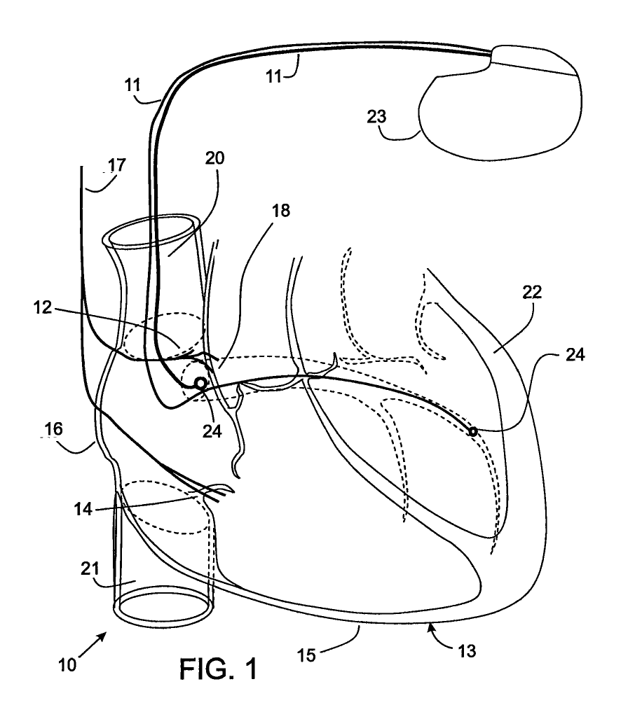

Reference FIG. 1, a medical device 10 is provided for artificially stimulating internal tissue, such as a heart 13 of a patient, by means of a stimulator 23 adapted for implantation in the patient. A plurality of stimulation leads 11 connect electrodes 24 to the stimulator 23 for sensing electrical signals in the heart and for applying electrical stimulation pulses to the heart tissue. The medical device 10 may stimulate the vagal nerve 14, 17 near the proximal coronary sinus (CS) 18 or from the inferior vena cava (IVC) 21 at the entry 12 into the right atrium 16, or from the superior vena cava (SVC) 20 at the entry 12 into the right atrium. During a treatment procedure, stimulation electrodes 24 are placed at locations near the vagal nerve 14, 17, such that one or more electrodes from a plurality of electrodes are programmably selected for optimal vagal stimulation. The stimulation waveforms are programmed with respect to envelope, shape, duration and duty cycle for maximizing energy conservation and minimizing stimulation sensation to patient. Atrial fibrillation sensing and stimulation further involves sensing right atrium (RA) 16 and right ventricle (RV) 15 or left ventricle (LV) 22 and detecting when RA rate is faster than RV or LV rate.

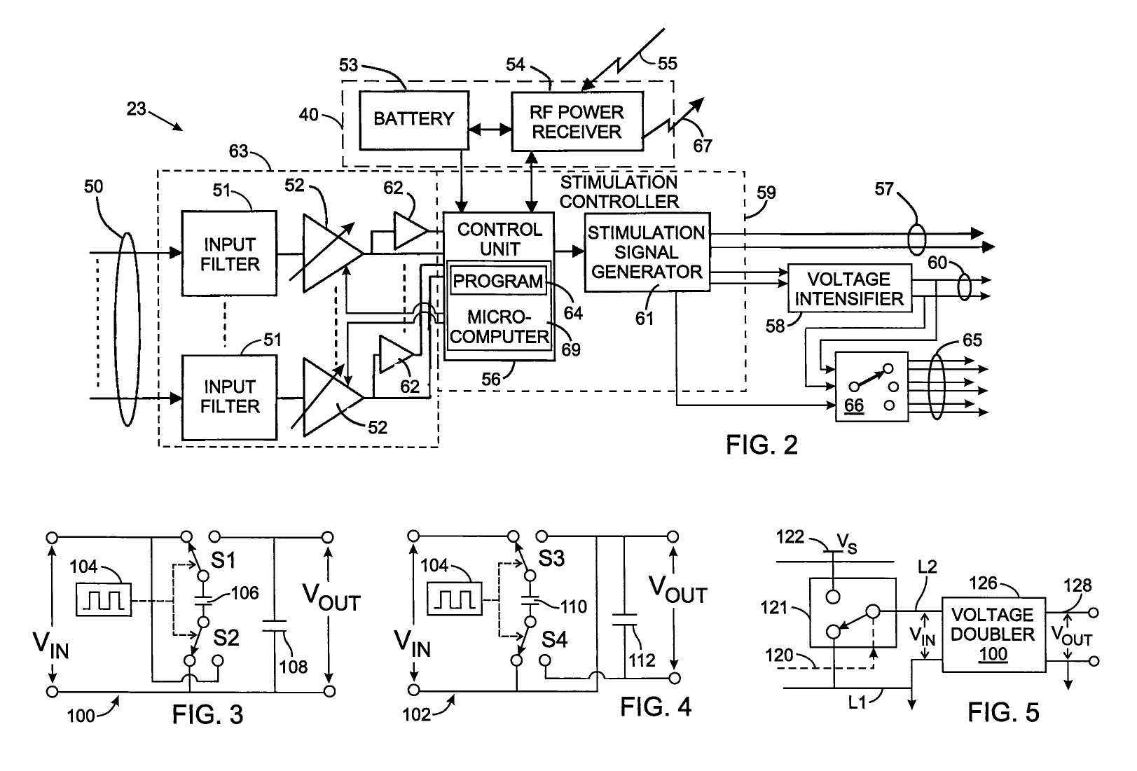

FIG. 2 schematically illustrates the circuitry in the stimulator 23. The stimulator 23 has a low impedance power supply 40 that comprises a battery 53 and a radio frequency (RF) transceiver 54 that derives electrical power from a received first wireless signal 55 for charging battery 53. As necessary, the first wireless signal 55 also carries control commands that specify operational parameters, such as the duration of a stimulation pulse or composition of a stimulation pulse that is applied to the output electrodes 57. Sensor electrodes 50 detect electrocardiogram signals and other physiological characteristics which are applied through input filters 51 to instrumentation amplifiers 52 of a sensing unit 63. The outputs of the amplifiers 52 are fed directly to a control unit 56 and through differential zero detectors (DZD's) 62 to the control unit. The control unit preferably is a computerized device that executes a software program which analyzes the signals from the sensor electrodes 50 to determine when to stimulate the patient's tissue, e.g. the coronary sinus vein or the right or left ventricle.

When stimulation is desired, the control unit 56 issues a command to a stimulation signal generator 61, which are both part of a stimulation controller 59. Depending upon the desired treatment, the stimulation signal generator 61 applies an electrical pulse directly to a first set of output electrodes 57 or drives a voltage intensifier 58 to apply a more intense stimulation pulse to a second set of electrodes 60. The voltage intensifier 58 may use any of several techniques to increase the stimulation pulse voltage from the standard low voltage implant battery 53, e.g. a three volt battery, contained within the implanted stimulator 23. Preferably, flying capacitor type voltage doubling, bipolar mode doubling, or a combination of both is used, as will be described. The stimulation leads with plurality of electrodes are designed to be a very low impedance structure to minimize power losses in the leads.

By monitoring the physiological response in response to the stimulation from either output electrodes 57 or 60, a feedback loop is formed which can be used to optimize the treatment or therapy.

### Stimulation Signal Regulation

The software executed by the control unit 56 analyzes the electrocardiogram signals and other physiological characteristics from the sensor electrodes 50 to determine when to stimulate the patient's physiology according to the algorithm executed by the control unit 56. The output pulses from the stimulation signal generator 61 can be applied either directly to output electrodes 57 or via an optional voltage intensifier 58 to electrodes 60 for high voltage stimulation.

The voltage intensifier 58 preferably is a “flying capacitor” inverter that charges and discharges in a manner that essentially doubles the power. This type of device has been used in integrated circuits for local generation of additional voltage levels from a single supply. FIGS. 3 and 4 respectively illustrate the doubler and inverter stages 100 and 102 of the voltage intensifier 58. In the doubler stage 100, a pair of switches S1 and S2 are operated by a square wave signal from a generator 104 to alternately charge and discharge an input capacitor 106 with the input voltage VIN. When the switches S1 and S2 are positioned as shown, the input capacitor 106 is charge by the input voltage VIN. During the discharge part of the switch cycle, the voltage across the input capacitor 106 added to the voltage already across an output capacitor 108, that is connected between the output terminals of the doubler stage 100. In the inverter stage 102 of FIG. 4, a second pair of switches S3 and S4 are operated by the square wave signal from the generator 104 to alternately charge and discharge an input capacitor 106 with the input voltage VIN to the inverter. During the discharge part of the switch cycle of this circuit, the voltage on the input capacitor 106 is applied across the output capacitor 112 and the output terminals in a manner that inverts the polarity of the output voltage VOUT with respect to the input voltage VIN. A doubler stage 100 and an inverter stage 102 can be connected in series to produce an increased inverted output voltage to apply to a pair of output electrodes 60. When there are more than two output electrodes 65, a switching circuit 66 is provided to selectively apply the output of the voltage intensifier 58 across one pair of those electrodes in order to stimulate a particular region of the physiology. This selection is controlled by the control unit 56. Various numbers of doubler stages 100 can be concatenated to increase the voltage from the stimulation signal generator 61 to the desired stimulation voltage level. The number of doubler stages may be switchable in response to control signals from the control unit 56, thereby enabling the voltage to be increased by different factors of two and inverted without use of inductors.

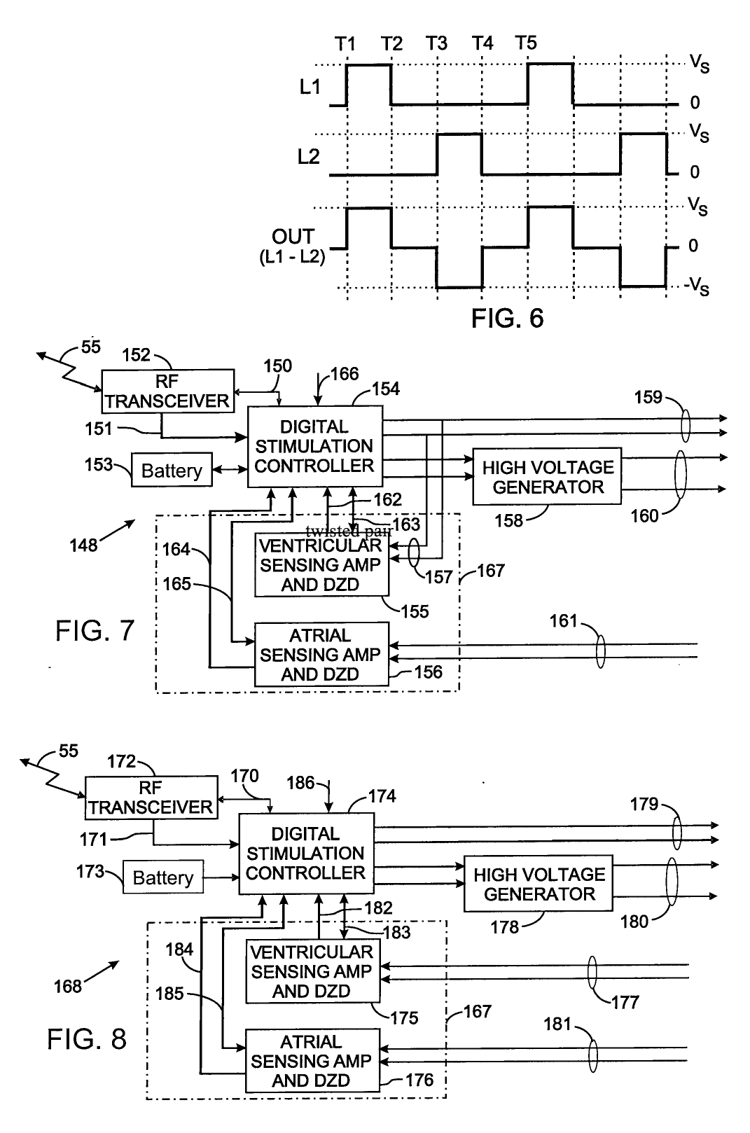

FIG. 6 depicts the signals on output lines L1 and L2 from either the stimulation signal generator 61 or intensifier 58. Bipolar operation is depicted in which both output lines L1 and L2 are switched between the supply voltage Vs and a reference potential, arbitrarily defined as the zero volts. In other words, each output line is switched between zero volts and Vs. Initially at time T0, both output lines L1 and L2 are connected to the zero volt reference potential. At time T1, the output line L1 is switched to the positive level Vs while output line L2 remains connected to the zero level, thereby rendering L1 positive with respect to L2 by Vs. Then at time T2, output line L1 is switched to the zero level, making the difference between L1 and L2 equal to zero. Next at time T3 output line L2 is switched to Vs while output line L1 remains at zero, thereby rendering L2 positive with respect to L1 by Vs. At time T4, both output lines are returned to zero and the voltage difference again is zero. The switching pattern T0 through T4 repeats successively beginning again at time T5. The switching produces a waveform designated OUT across the two output lines and the peak to peak voltage is twice the supply voltage Vs.

Thus several mechanisms provide stimulation pulses over a wide range of voltage levels. First by using single or double driven signals, as described above and shown in FIG. 6, an amplitude of one times Vs is available by keeping one line, e.g. L2, at zero at all times and switching L1 between zero volts and the supply voltage Vs. Double the supply voltage Vs is available by alternately switching both output lines L1 and L2. In addition, the output amplitude of one or two times Vs can be intensified or doubled by using a voltage doubler stage 100 (FIG. 3) or an inverter stage 102 (FIG. 4), thus allowing 1 Vs, 2 Vs, 4 Vs or higher to be created. Furthermore, bipolar output is available by using the inverter stage. This allows for 1 Vs, 2 Vs, 4 Vs or −Vs, −2 Vs or −4 Vs amplitude range. For example, with a 3 Volt battery 53, this technique permits output swings of 24 volts peak to peak between output lines, with one doubler and 48 volts by adding one inverter stage on each output line. The inherent low loss of using digital waveforms, rather than analog waveforms, which imply a dissipating element, the digital waveforms allow for conventional pulse width modulation as well, which would offer a duty cycle based method of achieving arbitrary values. However, for physiological stimulation, a more effective method is offered here, which makes use of a preamble to sensitize the nerve or tissue, followed by a low amplitude pulse body shown in FIG. 11, rather than a pulse which maintains full amplitude as shown in FIGS. 10A or 10B.

Determination of the voltage level, shape, and duty cycle of stimulation pulses shown in FIGS. 10A, 10B, and 11, which are applied to the sets of output electrodes 57, 60 or 65, is made by the control unit 56 in response to physiological characteristics detected by sensor electrodes 50 in FIG. 3. The output electrodes 57 also can be used for sensing to provide feedback signals for regulating the stimulation. For that purpose, the sensor electrodes 50 are connected to inputs of a variable gain instrumentation amplifier 52, shown in detail with respect to amplifier 407 in FIG. 15, that has an output coupled to an analog input of the control unit 56. The output signal from the instrumentation amplifier or amplifiers 52 also is applied to an input of a differential zero detector (DZD) 62 in FIG. 3. The DZD 62 performs signal transition detection and provides an output to the control unit 56 that tracks time events in the sensed physiological data signal to determine its rates and frequencies.

When stimulation is occurring, the instrumentation amplifier 407 in FIG. 15 has low gain (1× or less) to avoid saturation. When stimulation is inactive (high impedance across electrodes 412 and 413) as occurs between heart beats, the instrumentation amplifier 407 has a normal high gain (100×-200×) to sense physiological characteristics. The gain change is programmably achieved by commands via 405 from the control unit 56 sent to a control port of the instrumentation amplifier. The low gain setting allows measurement of the tissue and electrode interface impedance by using the known stimulation pulse duration and amplitude as a known source and the system impedance as a known impedance. From the sensed voltage and the known impedances, the tissue and electrode interface impedance can be determined. This information can also be logged over time to monitor physiological changes that may occur.

For stimulation verification, the control unit 56 analyzes the sensed parameters to calculate the actual heart rate to determine whether the heart is pacing at the desired rate in response to the stimulation. If the heart is being paced at the desired rate, the control unit 56 can decrease the stimulation energy in steps until stimulation is no longer effective. The stimulation energy then is increased to ensure maintenance of stimulation capture, thus ensuring a minimal excess of energy is being used. In common devices this threshold is set periodically in a doctor's office during a patient's visit. The proposed method is to automatically and continuously adjusts this for maximum efficiency, but only using as much energy as is needed, by constantly monitoring treatment efficacy. Energy reduction can be accomplished at least in two ways: (1) preferably, the duration of the stimulation pulse is reduced to linearly decrease that amount of energy dissipated in the tissue, or (2) the voltage amplitude is reduced in situations where energy dissipation might vary non-linearly because the tissue/electrode interface is unknown.

The stimulation is controlled by a functionally closed feedback loop. When stimulation commences, the sensed signal waveform can show a physiological response confirming effectiveness of that stimulation pulse. By stepwise increasing the stimulation pulse duration (duty cycle), a threshold can be reached in successive steps. When the threshold is reached, duration can be extended to provide a level of insurance that all pacing will occur above the threshold, or it may be sufficient to hold the stimulation pulse duration at the threshold. Again ensuring the limiting excess stimulation energy to a minimum and thus optimizing stimulation efficiency.

After each successful stimulation pulse, a determination is made regarding the difference in duration existing between the last non-effective pulse and the present effective pulse. That difference in duration is added to the present time. The system then senses the effectiveness of subsequent stimulation pulses and remains at the same level for either an unlimited duration or backs off one step in pulse duration. When the effectiveness is maintained again after a preset time window, which could be a number of beats, minutes or hours, the system backs off one decrement at a time. As soon as the effectiveness of the stimulation pulses is lost, the system increases the stimulation pulse duration until an effective pulse is obtained. In summary, the sensing and stimulation is a closed loop system with two feedback responses: the first response is following an effective pulse and involves gradual reduction of duration after a predetermined number of beats or a predetermined time interval; and the second response is to an ineffective pulse and is immediate with pulse duration adjustment occurring within one heart beat.

### Medical Device Configuration

FIG. 2 shows an exemplary configuration of an implanted device containing the element for efficient stimulation. Input signals from the sensor electrodes 50 are processed by filters 51 and instrumentation amplifiers 52 and presented to the control unit 56, as well as processed by the DZD 62. The control unit 56 contains an algorithm that interprets the received data and determines when stimulation is appropriate. The stimulation signal generator 61 is used to generate the digital waveforms and to control the voltage intensifier 58, if needed. The stimulation signals are carried to the physiological stimulation site, or in the case of higher (10 . . . 50V) stimulation, the output electrodes 60 from the intensifier 58 are used via a separate lead.

It is possible to combine stimulation output signals and use a single lead. However, the standard low voltage (2 . . . 5V) stimulation usually is used for muscle sites, whereas the higher (10 . . . 50V) signals are used to transvascularily stimulate nerve sites, such as the vagal nerve across the IVC (inferior Vena Cava) or CS (Coronary Sinus) fatpads.

Accordingly, the embodiment shown in FIG. 2 employs a low impedance power supply 40 that supplies energy to a control unit 56. The control unit controls the stimulation signal with a digital output delivered by the sets of output electrodes 57 or 60 to the stimulation site. The control unit also accepts input from the physiological signals, via the sensor electrodes 50, filters 51 and instrumentation amplifiers 52. The inputs do not use grounding and both input lines float with respect to ground, in order to minimize common mode noise. The sensing inputs are connected to the tissue through a very low input impedance (<50 Ohm) lead assembly with a two or more electrodes. Purpose specific segmented waveforms are delivered to the electrodes by the control unit. The control unit 56 delivers segmented digital waveforms shown in FIGS. 11, 12A, 12B that have voltage amplitude chosen to be close to the desired output voltage. In such a device, capture threshold is managed by modifying the duration of the output waveform thereby minimizing energy losses at the output stage. The segmented, stimulation waveforms may pass through a voltage intensifier 58 based on a specific stimulation purpose. As an example of an application requiring voltage intensification, an atrial fibrillation treatment device using vagal stimulation may require a high voltage (10-40 V) and at 20 to 200 Hz stimulation frequency. An example for an application that does not require voltage intensification is a pacing device to treat bradycardia that may use a relatively low voltage (2-5 V) and low rate (40-120 BPM or 0.67 Hz to 2.0 Hz)

An important aspect of the preferred embodiment is its capability to provide stimulation for two or more clinical purposes simultaneously. For example, the system can be configured to provide concurrent treatment for atrial fibrillation (Afib) and backup pacing to get the heart rate up in cases where the heart rate may fall below a predetermined rate. As another example, the system can be configured to provide concurrent atrial fibrillation and cardiac resynchronization therapy. The control for these functions would reside in the control unit 56 which would receive inputs from the sensor electrodes 50, while standard (2-5 Volt) stimulation output would be provided by either the stimulation signal generator 61 via the first set of output electrodes 57, or higher voltage (10-40 Volt) stimulation would be provided via the intensifier 58 the output electrodes 60.

Another embodiment of a stimulator 148, shown in FIG. 7. comprises a low impedance power supply 149, that supplies energy to a digital stimulation controller 154. The digital stimulation controller 154 governs production of the stimulation signal with a digital output delivered to the stimulation site. The controller also operates a sensing unit 167 that has electrical sensing devices 155 and 156 which do not have external grounding. The ventricular sensing amplifier and DZD 155 with inputs connected to the electrodes 159, the atrial sensing amplifier and DZD 156 with inputs connected to electrodes 161, and the digital stimulation controller 154 are connected to the patient's tissue through a very low input impedance lead assembly with a plurality of dynamically programmable electrodes. Purpose specific segmented waveforms are delivered to the electrodes by the digital stimulation controller. The digital stimulation controller 154 delivers segmented digital waveforms whose voltage amplitude is chosen to be close to the desired output voltage. A device capture threshold is managed by modifying the duration of the output waveform, thereby minimizing energy losses at the output stage. The segmented, stimulation waveforms may pass through a voltage intensifier stage or high voltage generator 158 based on a specific purpose. As an example of an application requiring voltage intensification or a high voltage generator stage, an atrial defibrillation device may require a high voltage (10-30 volts) at a 20 to 200 Hz stimulation frequency. As an example for an application that does not require voltage intensification, a pacing device to treat bradycardia may need a low voltage (2-5 volts) and stimulation low rate (40-120 BPM), equivalent to a frequency of (0.67 Hz to 3 Hz). The high voltage generator 158 is connected to the target stimulation site by means of a lead assembly with a plurality of electrodes 160 that are shared with the output 157 from the digital stimulation controller 154.

FIG. 8 depicts another stimulator 168 with essentially the same configuration of as the one in FIG. 7, but differs in that the high voltage generator output electrodes 180 are independent of the electrodes 177 coupled to the ventricular sensing amplifier and DZD 155, in the event that the optimum pacing site differs in location from the high voltage stimulation site.

The stimulators 148 and 168 in FIGS. 7 and 8 also sense a physiological characteristic of the patient and send related data via a wireless signal. The sensing device has no external ground and the input lines float with respect to ground, thereby being practically immune from noise sources that are inevitable in externally grounded devices. Employing ungrounded physiologically sensing provides a relatively noise free input signal, that is far more immune to electromagnetic interference (EMI) than conventional stimulation systems, which are affected by 50 Hz, 60 Hz and 400 Hz line frequencies, theft alarms, broken leads, etc. Because of this noise immunity, the stimulator 23 is less unlikely to misinterpret electrical noise as fibrillation and apply stimulation the patient. In addition to the discomfort to the patient, erroneous stimulation drains the battery and over time reduces the useful service life of the battery. In the case of a pacer, misinterpreted sensing information may yield an incorrect pacing rate or pacing when such is not required, again a loss of energy.

The output line 162 of the ventricular sensing amplifier and DZD 155 and the output line 164 of the atrial sensing amplifier and DZD 156 result from analysis by a derivative zero detector (DZD) shown in FIGS. 17B or 18B). The analysis further discriminates between noise 711 (FIG. 18A) from biological signals. The stimulation may be further adapted based on the analysis performed by the digital stimulation controller 154 to optimize stimulations.

These stimulators 148 and 168 have a capability to provide stimulation for two or more clinical purposes simultaneously. For example, the medical device 10 can be configured to provide concurrent treatment for atrial fibrillation and backup pacing to increase the heart rate in cases where the heart rate falls below a predetermined rate. As another example, the medical device 11 can be configured to provide concurrent atrial defibrillation and cardiac resynchronization therapy.

In the subsequent paragraphs, each of the modules of the stimulation system is described in detail.

### Power Supply:

Referring again to the embodiment in FIG. 2, the implanted stimulator 23 may be battery powered and thereby include a battery 53. The stimulator 23 also may derive electrical power from the energy of the first wireless signal 55 received by the transceiver 54. Alternatively the power supply can be based on other forms of energy supply including but not limited to piezoelectric devices, thermal energy sources, mechanical energy sources, and chemical energy sources. In some version, two or more of the energy sources are combined to supply power to the medical device. In any case, it is very important for overall efficiency to have an energy source with a low source impedance to minimize energy loss within the source itself.

### Control Unit:

The control unit 56 stores the operational parameters, which are either preprogrammed or received via the first wireless signal 55, for use in controlling operation of a stimulation signal generator 61 that applies tissue stimulating segmented voltages pulses across the sets of output electrodes 57, 60 and 65. Preferably, the control unit 56 comprises a conventional microcomputer 69 that has analog and digital input/output circuits and an internal memory that stores a software control program 64 and data gathered and used by that program.

The control unit 56 also receives signals from a plurality of sensor electrodes 50 that detect electrical activity of an organ in the patient. When that organ is the heart, the electrodes sense conventional electrocardiogram signals which are utilized to determine when a stimulation therapy should occur. Additional sensors for other physiological characteristics, such as temperature, blood pressure or blood flow, may be provided and connected to the control unit 56 via amplifiers, if needed. The control unit 56 stores a histogram of pacing data related to usage of the medical device 10 and other information which can be communicated via a second wireless signal 67 to a device external to the patient.

### Segmented Waveforms and Low Impedance Lead:

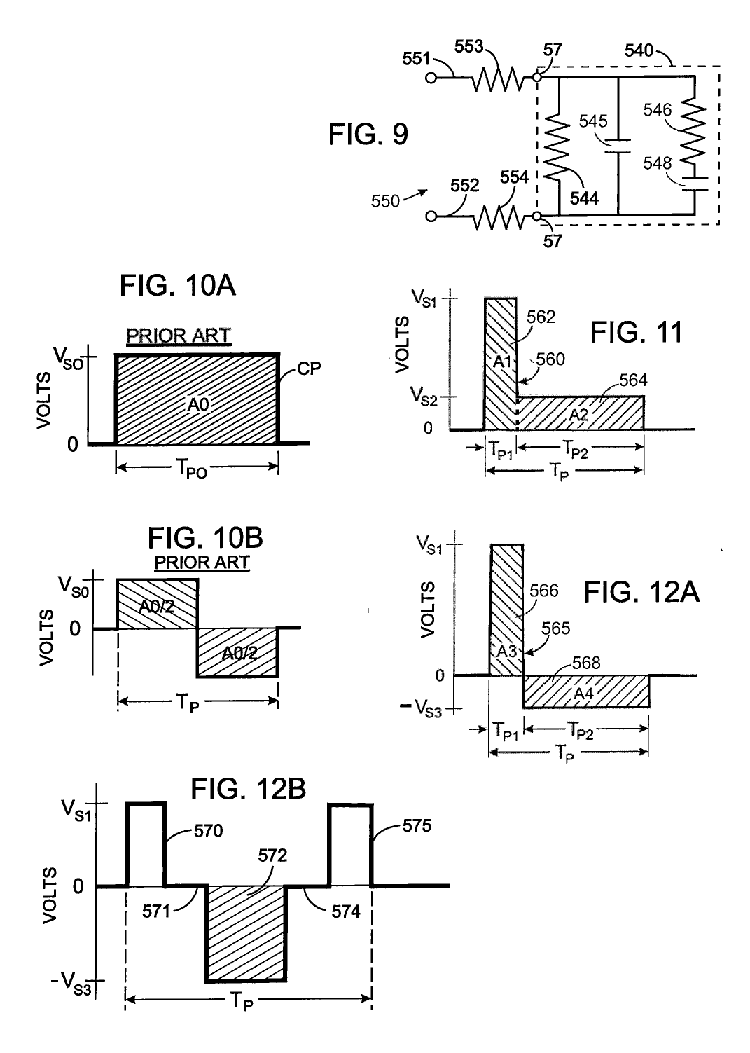

A novel ultra low resistance pacing lead circuit may be used in the present stimulation system. In FIG. 9, the pacing lead circuit 550 has first and second conductors 551 and 552, the combined resistance of which is less than 100 ohms, and preferably is less than ten ohms. Specifically, each conductors 551 and 552 has a aggregate impedance 553 and 554, respectively. The electrical characteristics of the tissue 540 being stimulated are modeled as an equivalent resistance 546 in series with an equivalent capacitance 548 that are in parallel with capacitance 545 and resistance 544. Since the tissue impedances are more or less given and fixed from patient to patient, lowering the lead impedance 553 and 554, has the effect of lowering the overall RC time constant for a signal presented to the lead circuit 550 and affect ultimate stimulation of the tissue 540 across the lead circuit 550. As a result, the circuit of the pacing lead circuit 550 has a significantly smaller RC time constant, which consequently speeds up the rise time of the stimulation pulse.

Upon activation of the stimulation system, the control unit 56 begins executing control program 64 that determines when and how to stimulate the patient's tissue. The control unit 56 receives signals form the from the sensor electrodes 50 that indicate the electrical activity of the heart or other body organ and analyzes those signals to detect irregular or abnormal activity. In response to detecting such activity, a command is sent to the stimulation signal generator 61 which causes that latter device to apply an electrical voltage pulse across the stimulation electrodes 57.

The waveform of that electrical voltage pulse, referred to as a composite pacing pulse, is illustrated in FIGS. 11, 12A and 12B. The composite pacing pulse 560 in FIG. 11 is characterized by a first segment 562 and a second segment 564 contiguous with the first segment, and preferably immediately following the first segment as illustrated. Both of the first and second segments 562 and 564 have rectangular shapes with the understanding that in actuality a rectangular pulse has leading edge that does not have an infinite slope and thus has a non-zero rise time. Similarly to clarify, the trailing edge of the first segment 562 also has a non-zero fall time. Specifically, the first segment 562 has a fast rise time (4V/μs); a duration between 0.005 ms and 0.5 ms, preferably 0.2 ms and a similarly fast (4V/μs) fall time.

The amplitude V_{S1} of the first segment 562 is at least three times greater than the amplitude V_{S2} of the second segment 564. The second segment 564 has a significantly longer duration T_{P2}, e.g., at least three times the duration T_{P1} of the first segment 562. The integral of the first segment 562 is graphical depicted by area A1 under that segment of the pulse, and integral of the second segment 564 is depicted by area A2. Preferably, the integral of the first segment 562 is substantially equal to the integral of the second segment 564.

The amplitude V_{S1} of the first segment 562 of the composite pacing pulse is at least three times greater than the nominal amplitude V_{S0} of the conventional pulse CP in FIG. 10A, while the second segment 564 has an amplitude V_{S2} that is less than that conventional pulse. The total duration T_{P0} of the composite pacing pulse 560 is less than the nominal duration T_{P0} of the conventional pacing pulse CP in FIG. 10A. The sum of the integrals (A1+A2) for the first and second segments 562 and 564 is less than the integral A0 of the conventional pacing pulse CP. Further note that efficiency is gained by expending less overall energy and clinical efficacy is gained by reducing the stimulation threshold for most of the pulse duration.

FIG. 12A illustrates an alternative composite pacing pulse 565 which is characterized by a fast rising, short duration, high positive amplitude first segment 566 that is substantially identical to the first segment 562 of the previously described composite pulse 560 in FIG. 11. However, the first segment 566 is followed by a different second segment 568 consisting of a negative voltage with an absolute amplitude that is equal to or less than one-third the absolute amplitude of the first segment 566. The duration T_{P2} of the second segment 568 is a significantly longer than, e.g. at least three times, the duration T_{P1} of the first segment 566. Here too, the integral A3 of the first segment 566 is substantially equal to the integral A4 of the second segment 568. Consequently, the sum of those integrals (A3+A4) is less than the integral (A0) of the conventional pacing pulse CP in FIG. 10A.

It should be noted that in contemplated embodiments, the waveforms may be biphasic, triphasic, or multiphasic with pauses (zero amplitude sections) between the segments. An exemplary triphasic waveform is illustrated in FIG. 12B. The stimulation cycle starts with a positive wave segment 570 having an amplitude of V_{S1}. It is followed by a zero volt pause segment 571. Segment 572 is a negative wave segment of amplitude −V_{S3}. Next is a pause segment 574 of 0V amplitude that is followed by a positive segment 575 of amplitude V_{S1}. It should be noted that if charge balancing is required at the stimulation site, the sum of the integral of positive segments should be set equal to the sum of the integral of negative segments. The amplitude of the positive and negative segments may or may not be equal. An example sequence with charge balance may have a 50 μs positive pulse segment, a 20 μs pause, 100 μs negative segment, 20 μs pause, and a 50 μs positive segment, with equal absolute amplitudes for the positive and negative segments. The notation used here for the representation of the waveform sequence is “+” for positive segments, “0” for the pause and “−” for the negative segments. This gives a sequence of “+, 0, −, 0, +” with a total of 100 μs positive, and 100 μs negative pulse segments. Another example sequence is 50 μs positive, 20 μs pause, 50 μs negative “+, 0, −”. Yet another example of a sequence with more than 3 phase segments is “+, 0, −, 0, +, 0, −, . . .”. Sequences such as these are more efficient in delivering stimulation compared to conventional waveforms shown in FIGS. 10A and 10B. By modifying the lobes and pauses, the stimulation effect can be optimized and tuned dynamically by the control unit 56 in order to maximize the battery power utility. It should be further noted that these segmented waveforms can be a part of a continuous stimulation regimen wherein the tissue is stimulated by the predetermined composite waveform sequence at intervals determined by the period of the stimulation frequency. In certain applications the stimulation may be command driven such that the stimulation is applied only if certain stimulation criterion that is programmed in the control unit is met. In such cases the digital stimulation waveforms would pause for a command from the control unit before applying a segmented, composite stimulation waveform at a tissue location.

The height and width of the segments, or lobes, of the stimulation waveform can be set dynamically by the stimulator 23. With reference to FIG. 2, upon implantation of the stimulator, the control unit 56 sets the voltage level Vs for the stimulation pulses. To do so, the control unit 56 commands the stimulation signal generator 61 to apply a relatively low voltage pacing pulse to the electrodes 57. Thereafter, the control unit 56 examines the input signals from the sensing unit 63 to determine whether that stimulation waveform caused contraction of the patient's heart. Typically the initial voltage level will be insufficient to trigger contraction. Thus the control unit 56 commands production of another pacing pulse at a higher voltage level. This process continues until the pacing pulse produces contraction of the patient's heart, at which time, the associated voltage level is stored as the level to use for subsequent pacing. This voltage level remains constant as that is the level required to overcome the insulating barriers formed by different tissue types through which the stimulation current has to flow and the impedance of those barriers does not change.

Occasionally the control unit 56 enters a configuration mode in which the duration of the segments of the pacing pulse are set. If the stimulator already has been configured, the control unit 56 repeatedly decreases the duration of the pacing pulse until capture does not occur, i.e., the pacing pulse does not produce a heart contraction. During this mode, the different lobes of the pacing pulse are decreased by the same proportion, however the width of each lobe could be varied independently. The pulse duration at with capture is lost is stored as the lower boundary of a duration window. Then, the control unit 56 repeatedly commands the application of pacing pulses that increases in duration until capture occurs. Typically, capture reoccurs at a pacing pulse having a longer duration that the pacing pulse at which capture was lost. The duration of the pulse when capture reoccurs is stored as the upper boundary of the duration window. Then the control unit 56 defines a duration setpoint as the average of the lower and upper boundaries of the duration window. Therefore, the present stimulator 23 maintains a constant stimulation voltage level, while compensating for physiological changes by adjusting the duration of the pacing pulse.

In some embodiments, the stimulated tissue may be cardiac muscle, or a nerve such as vagal nerve or a spinal nerve, bladder, brain, to name only a few. As mentioned earlier, in some embodiments, traditional devices such as pacemakers and defibrillators, pacemakers for vagal stimulation for atrial fibrillation therapy, and other types of pacers for bradycardia, resynchronization, vagal stimulation for central nervous system (CNS) conditions may benefit from the segmented composite stimulation waveforms.

### Sensing Circuit:

Referring again to FIG. 2, the sensing unit 63 includes an instrumentation amplifier 52 and a signal detector circuit or DZD 62. In the preferred embodiment, the sensing unit 63 is not connected to an external ground.

There are a few considerations in a practical implementation of the sensing unit 63. First, there are DC considerations. Second, there is an internal reference consideration. The third one involves filtering considerations.

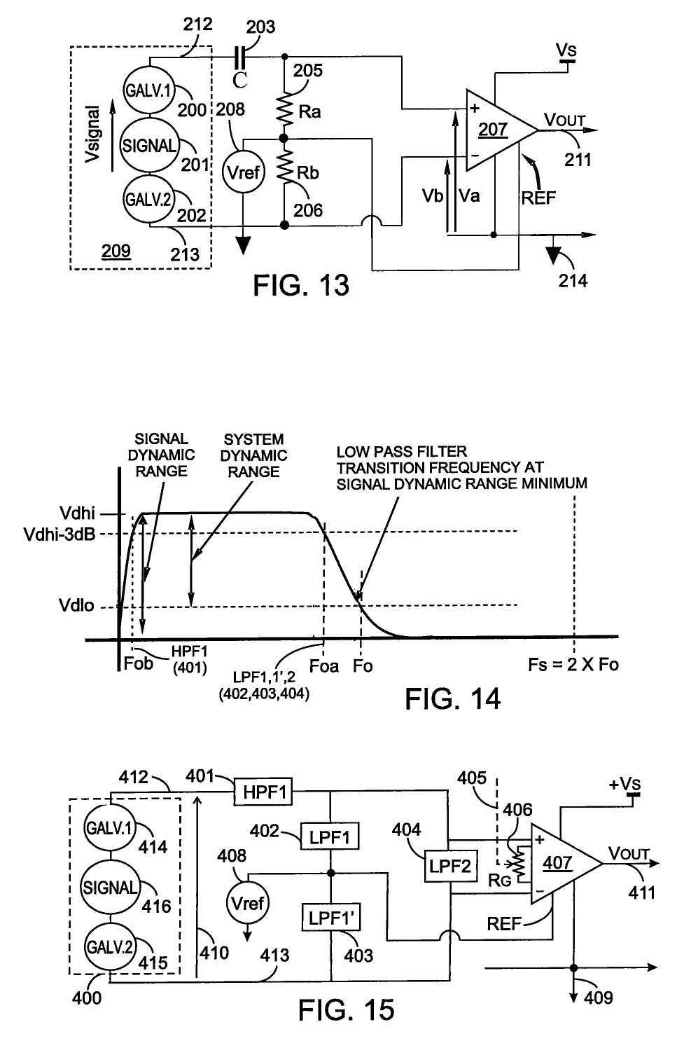

DC considerations: Referring to FIG. 13, in the physiological environment 209 at the interface between the electrodes and the patient tissue, a galvanic system is formed with a DC potentials 200 and 202. If there is complete symmetry in this circuit from a first electrode 212 to a second electrode 213, then all the contact potentials are cancelled rendering a zero net potential. However, if the materials used are dissimilar or have dissimilar temperatures, the electrode-tissue and or the electrode-blood interface yield different galvanic potentials that do not cancel. In this case, the input amplifier 207 is presented with the source voltage 201 along with the galvanic potential difference. This galvanic component is normally relatively static, but potentially may be modulated with body or organ movement, if the electrode wanders between touching the vessel wall and the blood pool thereby presenting a varying “DC” voltage. The variance over time is expected to be synchronous with the movement, and thus in the sub 2.0 Hz range, if respiratory and cardiac movements are included. Another DC issue stems from the amplifier itself, which will require a DC current bias into or out of the amplifier input terminals. In MOSFET amplifiers, this “bias current” is very small, but doubles with every 10° C. in temperature rise. Also, this current can have an offset, leaving a differential current that spoils the balance of a high impedance circuit. The solution to this problem is to provide a form of AC coupling capacitance 203 with the electrodes, and a DC current path, depicted by resistances 205 and 206, for the bias currents.

The AC coupling capacitance 203 performs two functions. The first function is DC decoupling from the galvanic voltages 200 and 202, and the second function is to form a high pass filter with a corner frequency of F_{HP}=½πRC, where R=Ra+Rb respectively resistances 205 and 206 and C is represented by capacitance 203.

The bias and offset currents are in the order of 10⁻⁹ to 10⁻⁸ A, and with path resistances of e.g. 100 kOhm, still yield 0.1 to 1.0 mV. Since source voltages are in order of 0.5-10 mV, these bias and offset voltages are not negligible. Therefore, in this invention, the amplifier specification selection should be such that these currents are low enough to allow for reasonably high input resistance values in the order of 100 kOhm or better for resistances 205 and 206 in FIG. 13.

Appropriate selection of values for resistances (Ra and Rb) 205 and 206 yields an acceptable low bias current offset voltage component (V_{offset}=I_{offset}×Ra, where Ra=Rb), and a proper F_{HP} (high pass filter frequency). The traditional corner frequency range for F_{HP} is in the order of 0.5 Hz to 2.0 Hz, but other values for capacitance 203 and resistances 205 and 206 can be selected depending on spectral regions of interest.

A natural feature that helps the implementation of the stimulator is the relatively low impedance of the patient tissues involved, typically 300 to 1200 Ohms between, for example, 5 mm spaced electrodes. Thus, in order to create a net 1.0 mV across such an impedance, energy density of 0.4 mW/m would be needed with the energy contained from 0-1 kHz.

Reference Considerations: In order to incorporate a floating AC coupled signal, such as shown in FIG. 13, it is desirable to provide a reference level V_{ref} 208. If the signal is expected to be symmetrical, V_{ref}=Vs/2 can be selected, thus allowing Vₒᵤₜ to swing between ground and Vₛ, with a “null” or rest point at V_{ref}. This reference voltage V_{ref} is provided to the output stage of the amplifier 207. Commercially available instrumentation amplifiers have a provision to receive reference input for the output stage. The original input signal 201 now can be presented at the output 211 as: Vₒᵤₜ=V_{signal}×Gain×F, where F is a high pass filter function formed by capacitance 203 and the sum of resistances 205 and 206.

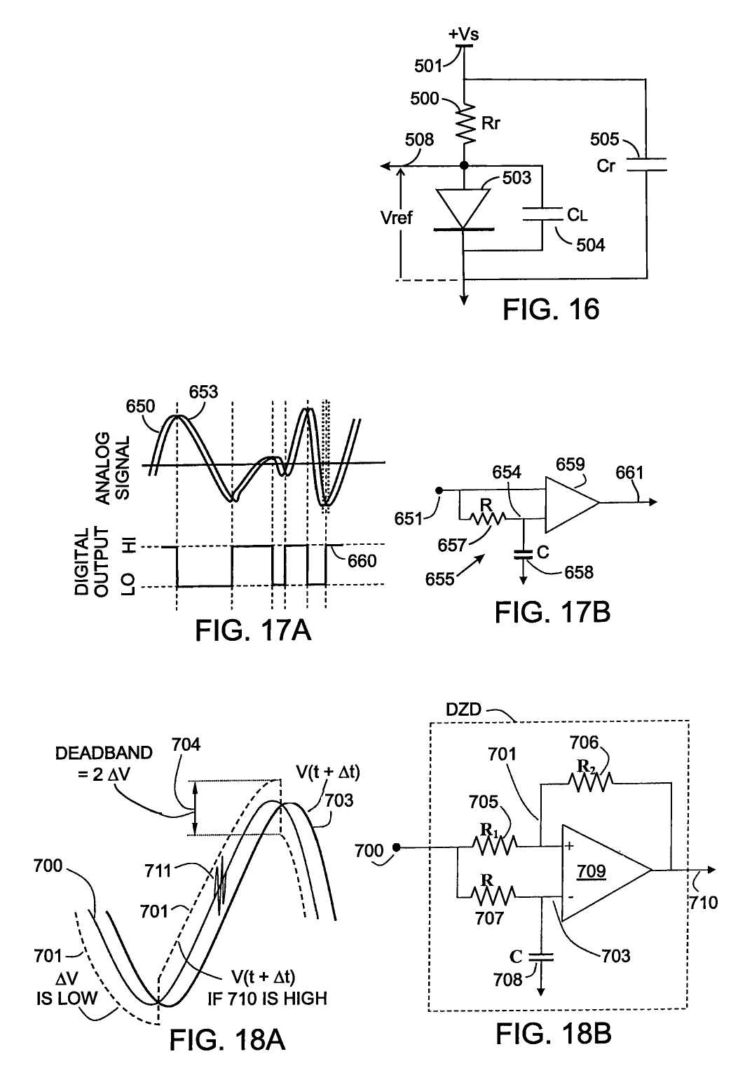

Additional details for the internal reference are provided in FIGS. 15 and 16. An example reference voltage of 1.2 V is achieved using a Gallium-Arsenic red light emitting diode (LED) 503 that has a series resistance Rᵣ 500 and parallel capacitances Cᵣ 505 and C_{L} 504. Two factors help in an LED serving as a stable reference voltage. First, the electronics module containing signal amplifier/detector as a part is in an intravascular environment, wherein the blood pool provides electromagnetic interference (EMI) shielding. Second, the thermal properties of this environment are relatively constant at the internal body temperature. Thus, it can be shown from the fundamental considerations that the voltage drop across the LED 503 remains sufficiently constant at a relatively constant temperature.

Filtering Considerations: Referring to FIGS. 14 and 15, if there is no meaningful information contained in the filtered out band, there will not be any adverse issues with the filtering approach. In practical applications, that is however rarely the case. Since important information is contained in those frequency bands, the current implementation is tailored to include the entire band from 10 Hz to 250 Hz. For robustness an even wider range of frequencies (e.g., 2 Hz 500 Hz) is used. With this consideration, the fast rise times of the cardiac muscle tissue signals containing high frequency content in the 100-250 Hz range can be easily accommodated in their pristine form. Additionally, by including these frequency components, the natural physiological signals can be easily distinguished from background signals, such as noise, voluntary and involuntary muscle movement etc.

The details of this aspect of invention are disclosed in FIGS. 14 and 15. Physiological environment 400 is shown to contain the galvanic voltages 414 and 415 formed at the tissue electrode intersections of two electrodes in an exemplary embodiment. The biological signal source 416 that would be sensed is shown as the signal generator with an associated voltage V_{signal}. The source may also have associated series source impedance (Z_{source}), which is not shown.

Between the biological environment 400 and the instrumentation amplifier 407, three filters 401, 402/403, and 404 are provided to perform various functions. The first is a high pass filter 401 that essentially blocks DC and low frequencies up to a prespecified cut-off (e.g., 2.0 Hz). This high pass filter 401 consists of passive elements with capacitance and resistance, where resistance may be obtained by a combination of resistors, and source impedance in series. The second filter, formed by filtering component filters 402 and 403, suppresses common mode noise by providing a suitable low pass filter (LPF1). This filter consists of passive elements C and R and their symmetrical counter parts (LPF1′).

The third filter 404 rejects high frequency noise signals by using a low pass filter (LPF2) formed by passive elements capacitor and resistors in series. Broadband ambient electromagnetic (EM) noise from appliances and other equipment could swamp the input circuit and consume dynamic range, and thus needs to be filtered out. In one approach, a low pass filter LPF2 with a cut-off at 1.0 kHz frequency is selected since the EM noise is broad band, but its energy is rather low below 10 kHz and can be effectively filtered out.

Other Considerations: For ECG signals obtained by direct connection to the cardiac venous vessel wall or muscle tissue, the signal path between the two or more input electrodes should exclude any electromagnetic pickup loop, for example, by twisting the lead and or wire pairs. Therefore, symmetrical layouts are favored.

In summary, as noted above, absence of a traditional ground in the stimulator 23 is a significant departure from the prior implanted stimulation devices and has obviated the need for notch filtering and other kinds of signal degrading processes. Another aspect of the invention as already mentioned is passive filtering at the front end, before any active components. As a result, physiological signals are obtained without any degradation. Finally, if used with an implanted device with a metal exterior housing, the sensing electrodes would not be in a circuit with the housing as in prior devices of that type since the metal housing is in contact with patient's tissues. The electronics, such as the battery, transceiver, amplifier, detector, filters, signal generator and control circuits normally are all contained within the metal exterior housing. In the traditional implementation that electrically conductive housing forms a ground or signal reference by contact with the patient's body. This in itself is a source of unbalanced noise and greatly diminished the overall ability of the system to resolve signals from EMI noise, such as line (50 Hz, 60 Hz, 400 Hz) noise as is commonly found in home, office and aircraft or naval equipment. In general, this is resolved by having notch filters for these frequencies, at the expense of losing great physiological signal frequency detail, as the physiological signals are contained within those same frequency ranges. Even a sharp (60 dB/decade) 50 Hz filter, will still attentuate significantly at 25 Hz or 100 Hz, thus creating frequency distortion. As a result, conventional stimulation devices have difficulty separating EMI noise from fibrillation. The in this embodiment described, groundless signal conditioning does not have these issues and demonstrates accurate signal representation, allowing for improved accuracy and efficacy in pacing or stimulation treatment.

Signal Detector: With reference to FIG. 2, the stimulation electrodes are connected to inputs of the variable gain instrumentation amplifiers 52 which have outputs coupled to analog inputs of the control unit 56. The output signal from each instrumentation amplifier 52 also is applied to a derivative zero transition detector (DZD) 62. Referring to FIGS. 17A, and 17B, the output signal from the instrumentation amplifier 52 is applied as an input signal 650 to an input 651 of DZD 655. The DZD 655 performs signal transition detection and provides an output signal 660 on line 661 to the control unit 54 (FIG. 2) that indicates of time events in the sensed physiological data signal 650.

In a preferred embodiment, the signal detector comprises a signal transition detector followed by an event classifier algorithm contained within the program of the control unit 56. The derivative zero transition detector 655 as shown in FIG. 17B includes a comparator 659, which is presented with the signal 650 at input 651 and a time shifted copy 653 of the signal (for example, a sinusoidal waveform) at another input 654. The comparator 659 identifies features in the input signal that are distinguished by having a local zero derivative representing the change of direction of the signal amplitude. The output signal 660 at the amplifier's output line 661 is a digital representation indicating signal direction change and time between these events.

The signal detector can be implemented using a circuit using conventional operational amplifiers for frequencies less than 200 Hz. However, for higher frequencies, comparator operational amplifiers are preferred to provide a digital output signal with well defined slopes. The method is sensitive to the time delay value, which will separate the signals in time. There are a number of conditions to consider in choosing the time delay value. It should prevent setting off events from small random noise amplitudes. It could be set to exclude certain portions of the cardiac signal time sequence. For example, when a good QRS signal is detected, a larger delay can be chosen.

In FIGS. 18A and 18B, it can be seen that the waveforms and the amplitude transition threshold (deadband) 704 needed to trip the comparator is a function of the associated hysteresis of the circuit, and the open loop gain of the comparator. The hysteresis amount ΔV is a function of the deadband that can be chosen based on the component selection. The resistors R₁ 705 and R₂ 706 are chosen such that their ratio approximates the desired hysteresis. The components R 707 and C 708 determine the time constant of the delay. The threshold required to switch states is a function of the gain and slew rate of the comparator or operational amplifier at the frequencies of interest. Typically the gain roll off rate is 20 dB/decade from 1 kHz onward. With such a roll off point, a 105 dB gain at 1 kHz reduces to a gain of 65 dB at 100 kHz. The slew rate is the maximum rate by which the output 710 can change state. For example, a 1 V/msec slew rate would require at least 5 ms to go from 0V to 5V, regardless how hard the input is being overdriven.

The output of the DZD 62 is a transformed signal which is discrete. It should be noted that this technique is immune to the variations in the input continuous signal unlike traditional methods. The discrete signal can be advantageously used for signal classification.

For example, the DZD 62 in conjunction with software executed by the control unit 56 determine the heart rate and use that information in an algorithm for pacing a patient's heart. The heart rate detection is based on the number of transitions counted over a predefined time interval. If the heart rate goes out of range for a given length of time and the frequency of the transitions remain in the non-fibrillation range, cardiac pacing can be initiated to pace the patient's heart. When the transition frequency indicates atrial fibrillation stimulation for atrial defibrillation can be initiated.

### Controlling the Sensing Circuit:

Referring to FIG. 2, when stimulation is occurring, the instrumentation amplifier 52 has low gain (1× or lower) to avoid saturation. When stimulation is inactive (high impedance across stimulation electrodes 57, 60 and 65) as occurs between heart beats, the instrumentation amplifier has a normal gain (100×-200×) to sense physiological characteristics. The gain change is programmably achieved by commands sent from the control unit 56 to a control port of the instrumentation amplifier 52. The low gain setting allows measurement of the tissue and electrode interface impedance by using the known stimulation pulse duration and amplitude as a known source and the system impedance as known impedance. From the sensed voltage and the known impedances, the tissue and electrode interface impedance can be determined. This information can also be logged over time to monitor physiological changes that may occur.

For stimulation verification, the control unit 56 analyzes the sensed parameters to calculate the actual heart rate to determine whether the heart 13 is pacing at the desired rate in response to the stimulation. If the heart is pacing at the desired rate, the control unit can decrease the stimulation energy in steps until stimulation is no longer effective. The stimulation energy then is increased until the desired rate is achieved. Energy reduction can be accomplished at least in two ways: (1) preferably, the duty cycle is reduced to linearly decrease that amount of energy dissipated in the tissue, or (2) the voltage amplitude is reduced in situations where energy dissipation might vary non-linearly because the tissue/electrode interface is unknown.

The stimulation is controlled by a functionally closed feedback loop. When stimulation commences, the sensed signal waveform can show a physiological response confirming effectiveness of that stimulation pulse. By stepwise increasing the stimulation pulse duration (duty cycle), a threshold can be reached in successive steps. When the threshold is reached, an additional duration can be added to provide a level of insurance that all pacing will occur above the threshold, or it may be sufficient to hold the stimulation pulse duration at the threshold.

After each successful stimulation pulse, a determination is made regarding the difference in duration existing between the last non-effective pulse and the present effective pulse. That difference in duration is added to the present time. The medical device 10 then senses the effectiveness of subsequent stimulation pulses and remains at the same level for either an unlimited duration or backs off one step in pulse duration. When the effectiveness is maintained again after a preset time window, which could be a number of beats, minutes or hours, the system backs off one decrement at a time. As soon as the effectiveness of the stimulation pulses is lost, the system keeps incrementing the duration until an effective pulse is obtained. In summary, the sensing and stimulation is a closed loop system with two feedback responses: the first response is following an effective pulse and involves gradual reduction of duration after a predetermined number of beats or a predetermined time interval; and the second response is to an ineffective pulse and is immediate with pulse duration adjustment occurring within one beat.

### Exemplary Clinical Application:

Having described the complete stimulation system, an exemplary clinical application can now be described to illustrate the utility of the stimulation technique.

Vagal Stimulation to Treat Atrial Fibrillation with Backup Pacing:

Atrial fibrillation rate control is carried out by stimulation of the vagus nerve near the proximal coronary sinus (CS). The literature on atrial fibrillation has demonstrated that it is a clinical possibility, however existing pacing systems cannot realistically perform atrial fibrillation treatment because of the energy that would be required for such stimulation with a continuous 20 Hz, 20 volt waveform. Using the current stimulation system, an efficient digital waveform based stimulation protocol consuming less energy makes atrial fibrillation treatment practical with a conventional pacemaker battery or an external RF power source. Additionally, segmented waveforms in conjunction low impedance leads and the flying capacitor voltage intensifier enable the desired therapy to be achieved. In one embodiment, atrial fibrillation treatment can be achieved in a metal housing, or “can”, such as is used for a traditional pacemaker. The modules of the stimulator 23 described previously permit compact implementation of this novel therapeutic device, which can be implanted in a similar manner as a traditional pacemaker.

An apparatus for vagal stimulation to treat atrial fibrillation using energy efficient digital stimulation system comprises two or more electrodes that are programmably selectable, waveforms that are programmably selectable, and the technique optimized to avoid ventricular fibrillation. The stimulation apparatus further comprises a backup pacemaker to raise the heart rate if it falls below a predetermined threshold during atrial fibrillation treatment.

During a treatment procedure, stimulation electrodes are placed near the vagal nerve in the patient, wherein one or more electrodes from a plurality of electrodes is programmably selected for optimal vagal stimulation. The stimulation waveforms are programmed with respect to shape, duration and duty cycle for maximizing energy conservation and minimizing stimulation sensation to patient. The atrial fibrillation sensing and stimulation further involves sensing left atrium (LA) and left ventricle (LV) and detecting when LA rate is faster than LV rate. This detection may be done by the DZD detector.

Programmable parameter initiates vagal stimulation based on LV heart rate. By setting an upper heart rate limit, vagal stimulation is employed when the limit is exceeded. It should be noted that in patients with known chronic atrial fibrillation, an atrial electrode may not be necessary and just the ventricular rate sensing may be used. This is also the case in other supraventricular tachycardias as well.

Ensuring patient safety during vagal stimulation: Atrial fibrillation treatment is characterized by high voltage (110 . . . 40 Volts) pacing from proximal coronary sinus (CS) location at 20 to 200 Hz. During this stimulation care needs to be exercised to ascertain that the LV not being paced from the CS location. This feature is needed because high voltage rapid pacing (such as 20 Hz stimulation) of the ventricle may induce a rapid life threatening ventricular arrhythmia. It is, therefore, desirable to confirm prior to such stimulation of the vagal nerve that the electrode has not unintentionally moved where the ventricle might be stimulated.

Safety can be ensured by one or more ways including controlling the rate of stimulation and real-time analysis of results of stimulation. From the stimulation control approach, high voltage pacing at more modest heart rates that are unlikely to induce life threatening arrhythmias may be used to confirm that the ventricle is not being stimulated. In an analysis-based approach, comparing morphology of electrograms from distal CS (LV) before and during pacing and noting that the morphology would not change if LV is not being paced. Further the heart rate detected from the LV would not be the same as the paced rate. A preferred method may utilize both stimulation and analysis approaches, wherein the heart is paced at rates near the ventricular rate prior to the vagal stimulation, and a comparison of the electrocardiogram before and after such pacing is performed. The comparison results would show no change in morphology electrogram if the ventricle is not being stimulated. Moreover, if pacing is performed at a rate moderately faster than the heart rate prior to vagal stimulation, the heart rate would not change if there was no stimulation or “capture” of the ventricular muscle.

Backup Left Ventricle pacing for vagal stimulation treatment: Backup left ventricle pacing is performed if heart rate becomes too slow due to excessive vagal stimulation. In order to protect the patient if the heart rate is excessively slowed (bradycardia) beyond a desired rate, for example 60 beats/min, demand pacing (pacing which occurs when a predetermined time interval passes without electrical activity) would occur and continue until the intrinsic heart rate exceeds a predefined lower limit rate. Note that the afore mentioned vagal stimulation with LV bradycardia pacing as a backup also may be used to reduce or obviate the need for medication.

As the previously, a high efficiency digital stimulation device enables a number of functionalities that improves upon existing techniques. By way of examples, one embodiment of such applications involves a bradycardia pacing treatment from an implanted stimulator 23. In this application, the high efficiency system provides extended battery life with fewer battery changes thereby resulting in less frequent surgeries. In another embodiment of clinical applications, a high efficiency device can improve the battery utilization since resynclronization pacing for congestive heart failure requires pacing devices to be used continuously. In addition, demand for power is higher for this application when compared to traditional bradycardia pacing since more sites need to be stimulated including both ventricles as well as the atrium. Again referring back to atrial fibrillation treatment, high efficiency may permit therapies now limited because of the relative inefficiency of prior art. As yet another example, in an intravascular stimulation system a higher efficiency permits longer times between recharging cycles and smaller intravascular storage components

In addition to the energy efficiency, robust sensing provides further advantages beyond the systems described herein. For example, in bradycardia pacing, robust sensing translates into less inhibition due to inappropriate tracking resultant from internal and external electromagnetic interference. In another example, for implantable cardioverter defibrillators, a robust sensing module may reduce the risk of inappropriate shock therapy resultant from EM interference or internal noise such as those that occur from lead fractures and defective header connections.

The foregoing description was primarily directed to a preferred embodiment of the invention. Although some attention was given to various alternatives within the scope of the invention, it is anticipated that one skilled in the art will likely realize additional alternatives that are now apparent from disclosure of embodiments of the invention. Accordingly, the scope of the invention should be determined from the following claims and not limited by the above disclosure.

What is claimed is:

1. An implantable apparatus for artificial electrical stimulation of tissue in a patient, the apparatus comprising:
a plurality of electrodes in contact with the tissue at one or more locations inside the patient; and
an implantable stimulator comprising:
(a) a power supply furnishing energy for the artificial electrical stimulation,
(b) a control unit for controlling the artificial electrical stimulation by programmable selection of at least some of the plurality of electrodes, and
(c) a stimulation signal generator connected to the control unit and producing a segmented stimulation waveform comprising a positive a segment, a pause segment, and a negative segment, wherein the segmented stimulation waveform is controlled by the control unit with respect to shape, duration and duty cycle.

2. The apparatus as recited in claim 1 further comprising a voltage intensifier connected to stimulation signal generator for increasing voltage of the segmented stimulation waveform to produce an output waveform that is applied to at least one of the plurality of electrodes.

3. The apparatus as recited in claim 2 wherein the voltage intensifier is one of a flying capacitor-type voltage doubler, bipolar mode voltage doubler, or a combination of a flying capacitor type and a bipolar mode voltage doubler.

4. The apparatus as recited in claim 1 wherein the segmented stimulation waveform is altered in response to a feedback signal received from a location inside the patient.

5. The apparatus as recited in claim 1 wherein the control unit adjusts duration of the segmented stimulation waveform by:
(a) decreasing the duration of the segmented stimulation waveform applied to a pair electrodes until the tissue fails to respond to such stimulation which determines a first duration value,
(b) thereafter increasing the duration of the segmented stimulation waveform applied to the pair electrodes until the tissue respond to such stimulation which determines a second duration value, and
(c) defining a duration setpoint that is between the first duration value and the second duration value, inclusive, wherein the duration setpoint is used in producing a subsequent segmented stimulation waveform.

6. The apparatus as recited in claim 1 wherein the segmented stimulation waveform has a first segment with a first amplitude, and a second segment that is longer in duration than the first segment and that has a second amplitude which is opposite in polarity and lesser in absolute magnitude to the first amplitude.

7. The apparatus as recited in claim 1 wherein the power supply is radio frequency based, piezoelectric device based, thermal energy source based, mechanical energy source based, or chemical energy source based.

8. The apparatus as recited in claim 1 wherein stimulation electrodes are located near a desired stimulation site.

9. The apparatus as recited in claim 8 wherein the stimulation site is selected from an intravascular location, a transvascular location, a muscle location, a transcutaneous location, and a nerve location.

10. The apparatus as recited in claim 1 wherein the control unit comprises a conventional microcomputer with analog and digital input/output circuits and an internal memory storing a software control program.

11. The apparatus as recited in claim 1 further comprising a sensing unit for detecting at least one physiological parameter from a site of stimulation.

12. The apparatus as recited in claim 11 wherein the sensing unit comprises an instrumentation amplifier with ungrounded, electrically floating inputs that receive signals from electrodes adapted to contact the tissue.

13. The apparatus as recited in claim 1 further comprising a sensing unit for detecting at least one physiological parameter, and comprising an electrode pair for implantation inside the patient, an instrumentation amplifier having an internal voltage reference, a passive filter coupling the electrode pair to the instrumentation amplifier.

14. An implantable apparatus for artificial electrical stimulation of tissue in a patient, the apparatus comprising:
a plurality of electrodes in contact with the tissue at one or more locations inside the patient;
a power supply furnishing energy for the artificial electrical stimulation,
an implantable control unit for controlling the artificial electrical stimulation by programmable selection of at least some of the plurality of electrodes, and by selection of a stimulation waveform;
a stimulation signal generator connected to the control unit and producing a segmented stimulation waveform that has a first segment with a first amplitude of a first polarity, and a second segment that is longer in duration than the first segment, wherein the second segment has a second amplitude which lesser in absolute magnitude that the first amplitude, and has a second polarity opposite to the first polarity.

15. The apparatus as recited in claim 14 further comprising a voltage intensifier connected to stimulation signal generator for increasing voltage of the segmented stimulation waveform to produce an output waveform that is applied to at least one of the plurality of electrodes.

16. The apparatus as recited in claim 15 wherein the voltage intensifier is one of a flying capacitor-type voltage doubler, bipolar mode voltage doubler, or a combination of a flying capacitor type and a bipolar mode voltage doubler.

17. The apparatus as recited in claim 14 wherein the stimulation waveform is altered in response to a feedback signal received from a location inside the patient.

18. The apparatus as recited in claim 14 wherein the segmented stimulation waveform is variable in shape, duration, and duty cycle for maximizing energy conservation and avoiding stimulation sensation to the patient.

19. The apparatus as recited in claim 14 wherein the power supply is radio frequency based, piezoelectric device based, thermal energy source based, mechanical energy source based or chemical energy source based.

20. The apparatus in claim 14 wherein the one or more locations inside the patient is one of an intravascular location, a transvascular location, a muscle location, a transcutaneous location and a nerve location.

21. An implantable apparatus for artificial electrical stimulation of tissue in a patient, the implantable apparatus comprising:
a plurality of electrodes in contact with the tissue at one or more locations inside the patient;
a power supply furnishing energy to the apparatus;
an implantable control unit comprising a microcomputer with analog and digital input/output circuits and an internal memory storing a software control program;
a stimulation signal generator controlled by the control unit and producing a segmented voltage waveforms across pairs of the plurality of electrodes to stimulate the tissue, wherein the segmented voltage waveforms are variable in shape, duration, and duty cycle.

22. The implantable apparatus as recited in claim 21 further comprising a sensing unit that comprises an electrode pair for implantation inside the patient to sense biological signals, an instrumentation amplifier having an internal voltage reference, a passive filter coupling the electrode pair to the instrumentation amplifier.

23. The implantable apparatus as recited in claim 21 further comprising a sensing unit for detecting at least one physiological parameter from a site of stimulation, wherein the sensing unit comprises an amplifier with ungrounded, electrically floating inputs that receive signals from electrodes at the site of stimulation.

24. The apparatus as recited in claim 21 wherein at least one of the segmented voltage waveforms has a first segment with a first amplitude of a first polarity, and a second segment that is longer in duration than the first segment, wherein the second segment has a second amplitude which lesser in absolute magnitude that the first amplitude, and has a second polarity opposite to the first polarity.
